(19) United States
(12) Patent Application Publication
(10) Pub. No.: US 2011/0136140 A1
(43) Pub. Date: Jun. 9, 2011

(54) DIAGNOSIS OF SYSTEMIC DISEASES

(76) Inventors: Péter Németh, Pécs (HU); Tamás Czömpöly, Dombóvár (HU); Tímea Berki, Pécs (HU); László Czirják, Pécs (HU)

(21) Appl. No.: 13/055,320

(22) PCT Filed: Jul. 21, 2009
(86) PCT No.: PCT/IB2009/053160

§ 371 Date: Jan. 21, 2011

(30) Foreign Application Priority Data
Jul. 21, 2008 (HU) P0800448

(51) Int. Cl. G01N 33/53; C12N 9/90

(52) U.S. Cl. 435/7.6; 435/7.1; 435/233

(57) ABSTRACT

It has been found that the pattern of recognized topo I epitopes is different between dcSSc, IcSSc and SLE patients. Fragment F4 (amino acid (AA) 450-600) was recognized by all patients tested. Fragment F1 (AA 5-30) and Fragment F8 (AA 350-400) represent characteristic epitopes for dcSSc and SLE, respectively. The invention relates to diagnostic uses and methods as well as kits for use in diagnosis.

This is the National Stage of International Application PCT/1B2009/053160, filed Jul. 21, 2009.

The present invention relates to novel diagnostic methods for systemic sclerosis and systemic lupus erythematosus, uses of anti-topoisomerase I and fragments thereof for such diagnosis and diagnostic kits.

### BACKGROUND ART

Systemic sclerosis (SSc) is a systemic autoimmune disorder characterized by immune activation, vascular injury, inflammation, fibrosis of the skin and various internal organs. Activation of the immune system leads to production of disease specific autoantibodies, lymphocyte activation and secretion of various cytokines [1]. The vast majority of SSc patients have antinuclear antibodies, which predominantly recognize DNA topoisomerase I (topo I or TOP I), RNA polymerases, centromere proteins, and U3RNP [2].

Topo I is a 765 amino acid (AA) long DNA-relaxing enzyme which contains five distinct regions: the N-terminal domain (AA 1-215), core subdomains I-II (AA 216-435), core subdomain III (AA 436-636), the linker domain (637-713) and the C-terminal domain (AA 714-765).

Clinically patients with SSc could be classified into two distinct subsets. dcSSc is characterized by extensive fibrosis of the skin, lungs and other internal organs, while in lcSSc vascular abnormalities are dominating and fibrosis is limited [5].

In addition to SSc presence of anti-topo I antibodies has been demonstrated in systemic lupus erythematosus (SLE) patients showing no clinical signs and symptoms of systemic sclerosis [6,7]. These findings suggest that the presence of anti-topo I antibodies could have heterogeneous clinical consequences.

Use of topo-I and topo-I peptides have been proposed in detection of autoantibodies.

In U.S. Pat. No. 5,070,192 cloning of a topo-I polypeptide is described and detection of autoantibodies in patient samples by binding to said topo-I polypeptide is suggested. In U.S. Pat. No. 5,849,503 a mutant topo-I polypeptide is described which is thought to be useful for detecting autoantibodies e.g. in an immunoassay.

Later, a number of groups have studied the epitope specificity of anti-topo I antibodies in SSc patients. In these studies, nevertheless, various epitopes in the central and C-terminal part of the molecule [15-20] have been suggested. Reports suggest that an immunodominant region of topo I spans AA 489-573 [15, 16, 19]. However, a study which used recombinant fusion proteins constructed on the basis of the domain structure of topo I demonstrated that the core subdomains I-II is recognized more frequently than core subdomain III [11]. Longitudinal analysis of anti-topo I auto-antibodies revealed that reactivity against these regions is stable [9, 11], though a study using a limited number of sera showed that the regions recognized by anti-topo I auto-antibodies vary over time [10]. Thus several uncertainties existed in the art regarding topo I epitopes in systemic diseases and while epitope specificity of anti-topo I autoantibodies has been studied by a number of groups, there is no report of comparative epitope mapping in patients with dcSSc, lcSSc and SLE and there is no suggestion for a differential diagnosis of these diseases based on autoantibody binding to different epitopes.

### BRIEF DESCRIPTION OF THE INVENTION

The present invention relates to a use of one or more isolated peptide(s) for diagnosis of a systemic autoimmune disorder, said one or more peptide(s) separately or simultaneously comprising at least one of the following epitopes:
a) an epitope having the immunological property of an at least 20 amino acids long segment of the portion of a DNA Topoisomerase I spanning amino acids 5 to 30,
b) an epitope having the immunological property of an at least 20 amino acids long segment of the portion of a DNA Topoisomerase I spanning amino acids 350 to 400,
   said one or more peptide(s) having an amino acid sequence which is at least 70%, 75%, 80%, 85% or 90%, 92%, 94%, 96% or 98% identical with the amino acid sequence of a peptide fragment of a wild type DNA Topoisomerase I,
   wherein
if binding of autoantibodies of a sample from a patient to epitope according to a) is detected, it is considered as indicative of diffuse cutane systemic scleroderma (dsSSC), preferably a late onset thereof, in said patient,
if binding of autoantibodies of a sample from a patient to epitope according to b) is detected, it is considered as indicative of systemic lupus erithematosus (SLE), preferably an SLE with Raynaud's phenomenon, in said patient.

In preferred embodiments, one or more isolated peptide(s) also comprise
c) an epitope having the immunological property of the at least 20 amino acids long segment of the portion of said DNA Topoisomerase I spanning amino acids 451 to 593, and wherein
if binding of autoantibodies of a sample from a patient to epitope according to a) and c) is detected, and preferably binding of autoantibodies of said patient sample to epitope according to b) is not detected, it is considered as indicative of diffuse cutane systemic scleroderma (dsSSC) in said patient or preferably a late onset thereof,
if binding of autoantibodies of a sample from a patient to epitope according to b) and c) is detected, and preferably binding of autoantibodies of said patient sample to epitope according to a) is not detected, it is considered as indicative of systemic lupus erithematosus (SLE) in said patient or preferably an SLE with Raynaud's phenomenon.

Preferably, the epitope of said peptide is different in not more than 10, 9, 8, 7, 6, 5, 4, 3, 2 or 1 amino acids from the respective epitope of the wild type sequence.

In a further preferred embodiment
epitope according to a) is an at least 20 amino acids long segment of the portion of said DNA Topoisomerase I spanning amino acids 5 to 30, and/or
epitope according to b) is an at least 20 amino acids long segment of the portion of said DNA Topoisomerase I spanning amino acids 350 to 400, and/or, preferably
epitope according to c) is an at least 20 amino acids long segment of the portion of said DNA Topoisomerase I spanning amino acids 451 to 593.

In a preferred embodiment all the epitopes are present on separate peptides.

In an embodiment peptides epitopes a) and b) are present on different peptides. In an embodiment peptides may be used wherein epitopes according to a) and c) are present on the same peptide and epitope according to b) is present on a different peptide, or epitopes according to b) and c) are present on the same peptide and epitope according to a) is present on a different peptide.

If binding of an antibody to an epitope can be detected specifically, in an alternative embodiment, epitopes according to a) and c) may be present on one peptide and epitopes according to b) and c) may be present on another peptide.

If desired, appropriate antibody standards are to be used.

In a further aspect the invention relates to a kit, preferably a diagnostic kit, for use in the diagnosis of a systemic autoimmune disorder, said kit comprising one or more peptides as defined in any of the previous claims,

said one or more peptide(s) separately or simultaneously comprising at least two of the following epitopes:
a) an epitope having the immunological property of an at least 20 amino acids long segment of the portion of a DNA Topoisomerase I spanning amino acids 5 to 30,
b) an epitope having the immunological property of an at least 20 amino acids long segment of the portion of a DNA Topoisomerase I spanning amino acids 350 to 400, and preferably
c) an epitope having the immunological property of an at least 20 amino acids long segment of the portion of a DNA Topoisomerase I spanning amino acids 451 to 593.

A preferred kit of the invention comprises
at least one peptide selected from
   a) a peptide comprising an epitope having the immunological property of an at least 20 amino acids long segment of the portion of a DNA Topoisomerase I spanning amino acids 5 to 30,
   b) a peptide comprising an epitope having the immunological property of an at least 20 amino acids long segment of the portion of a DNA Topoisomerase I spanning amino acids 350 to 400, and
a reference peptide comprising an epitope having the immunological property of an at least 20 amino acids long segment of the portion of a DNA Topoisomerase I spanning amino acids 451 to 593; and optionally
means for detecting binding of patient autoantibodies to a peptide epitope.

Preferably,
the peptide according to a) is a peptide fragment of said DNA Topoisomerase I spanning amino acids 5 to 30,
the peptide according to b) is a peptide fragment of said DNA Topoisomerase I spanning amino acids 350 to 400, and
the peptide according to c) is a peptide fragment of said DNA Topoisomerase I spanning amino acids 451 to 593.

In a further aspect the invention relates to a diagnostic method useful for differential diagnosis of systemic autoimmune disorders comprising the steps of
i) contacting a sample obtained from a patient with one or more peptide(s) comprising at least one of the following epitopes:
   a) an epitope having the immunological property of an at least 20 amino acids long segment of the portion of said DNA Topoisomerase I spanning amino acids 5 to 30,
   b) an epitope having the immunological property of an at least 20 amino acids long segment of the portion of said DNA Topoisomerase I spanning amino acids 350 to 400
      said one or more peptide(s) having an amino acid sequence which is at least 70%, 75%, 80%, 85% or 90%, 92%, 94%, 96% or 98% identical with the amino acid sequence of a peptide fragment of a wild type DNA Topoisomerase I,
      wherein
if binding of autoantibodies of said sample to epitope according to a) is detected, it is considered as indicative of diffuse cutane systemic scleroderma (dsSSC), preferably a late onset thereof, in said patient,
if binding of autoantibodies of said sample to epitope according to b) is detected, it is considered as indicative of systemic lupus erithematosus (SLE), preferably an SLE with Raynaud's phenomenon, in said patient.

In a preferred method of the invention, the one or more isolated peptide(s) also comprise
c) an epitope having the immunological property of the at least 20 amino acids long segment of the portion of said DNA Topoisomerase I spanning amino acids 451 to 593, and wherein
if binding of autoantibodies to epitope according to a) and c) is detected, and preferably binding of autoantibodies of said patient sample to epitope according to b) is not detected, it is considered as indicative of diffuse cutane systemic scleroderma (dsSSC) in said patient or preferably a late onset thereof,
if binding of autoantibodies to epitope according to b) and c) is detected, and preferably binding of autoantibodies of said patient sample to epitope according to a) is not detected, it is considered as indicative of systemic lupus erithematosus (SLE) in said patient or preferably an SLE with Raynaud's phenomenon.

Preferably the one or more peptide(s) as defined above are used in the methods, uses and or kits of the invention.

In a preferred embodiment the sample is a blood sample, preferably a serum sample.

### DEFINITIONS

“An epitope having the immunological property of a segment of a portion of DNA Topoisomerase I” is understood herein as an epitope capable of binding an antibody also capable of binding to said segment, preferably a patient antibody, more preferably a patient antibody of a patient having a systemic disease, preferably a systemic sclerosis or systemic lupus erythematosus.

“DNA Topoisomerase I” in accordance with the present invention can be a wild type DNA Topoisomerase I (EC 5.99.1.2.) from eukaryotic, preferably vertebrate, preferably mammalian, more preferably human source, or having the sequence identical with such a wild type DNA Topoisomerase I, or a mutant having a sequence identity of at least 50%, 60%, 70%, 75%, 80%, 85%, 90%, 93%, 95%, 97%, 98%, 99% or 100% therewith. Preferably at least one immunological property of said mutant DNA Topoisomerase I is maintained or one or more antibody capable of binding to the wild type protein is capable of binding to the mutant protein as well. Said DNA Topoisomerase I can be isolated from a natural source or recombinantly prepared.

The term “peptide”, as applied herein denotes any sequence of amino acids having an epitope for autoantibodies to eukaryotic, preferably vertebrate, preferably mammalian, more preferably human topoisomerase I (topo I). Preferably, the sequence of amino acids of said peptide is encoded by all or part of a polynucleotide, e.g. a cDNA, encoding said topo I or the amino acids sequence of said peptide having a sequence identity of at least 50%, 60%, 70%, 75%, 80%, 85%, 90%, 93%, 95%, 97%, 98%, 99% or 100% therewith.

“Autoantibodies” are antibodies of a subject raised against or capable of binding to a protein of said subject.

“Patient”, as used herein, refers to an animal or human subject who is treated, diagnosed or a body sample of whom is analyzed, or to be treated, diagnosed or analyzed.

### BRIEF DESCRIPTION OF THE FIGURES

FIG. 1 The pattern of recognized topoisomerase I (topo I) epitopes is different between dcSSc, lcSSc and SLE patients. Deduced amino acid sequences of phage clones selected with IgG purified from 5 diffuse cutaneous systemic sclerosis (dcSSc), 6 limited cutaneous systemic sclerosis (lcSSc) and 4 systemic lupus erythematosus (SLE) patients are plotted along the human topo I sequence.

FIG. 2 Recombinant topoisomerase I-maltose binding protein fusion constructs used in this study.

FIG. 3 Immunoblots using topoisomerase I (topo I) fusion proteins F4 and F1 as antigens. Purified recombinant fusion proteins (panel A: F4, panel B: F1) or maltose binding protein (MBP) were separated on a 10% SDS-polyacrylamide gel and transferred to nitrocellulose membranes. The membranes were cut into strips and probed with serial serum samples (obtained at dates indicated) of patients or with an anti-MBP antibody (a-MBP). MW: molecular weight marker (kDa).

### DETAILED DESCRIPTION OF THE INVENTION

The role of anti-topo I antibodies in pathogenesis of SSc is not fully understood, however, it can be assumed based on the present invention that immune response against topo I may differ among anti-topo I positive patients leading to production of anti-topo I autoantibodies with different epitope specificity.

In the prior art various recombinant topo I fragments were used, which were designed either on the basis of topo I domain structure or antigenicity prediction, both of which could miss possible epitopes [12-18]. For example, Hu et al. [21], who used a fusion protein covering the entire length (AA 1-213) of the N-terminal domain, showed that this part of the molecule is not targeted by anti-topo I antibodies. To the contrary, other previous studies performed with fusion proteins covering the N-terminal domain starting from AA 70 reported that this part of the molecule is recognized by anti-topo I antibodies [13, 16, 20].

These seemingly contradictory results may be due to the different methods and antigen constructs applied, and quite probably to possible conformational factors which could influence the accessibility of short epitopes buried in the tertiary structure. It is noted here in advance that the majority of new epitope-containing fragments, including fragment F1, identified by the present inventors at the N-terminal part spans only 20-30 AA.

Thus, the present inventors have chosen a different strategy and have constructed an antigen fragment library of topo I displayed on bacteriophage lambda and screened this library with sera of dcSSc, lcSSc and SLE patients. Regions of topo I selected from the library were expressed as recombinant fusion proteins and were further tested with patients' sera. Longitudinal analysis of epitope specificities has been performed and compared with clinical findings.

On the basis of fragments selected from the phage displayed antigen fragment library we expressed nine topo I-MBP fusion proteins and tested these fragments with 67 anti-topo I antibody positive patients' sera. According to our results recognition of the majority of fragments (F2, F3, F5-7, F9) is characteristic for the individual patient sera used for library screening, instead of being characteristic for the given disease subgroup. This is in agreement with result of Henry et al, who found both individual and longitudinal differences in the recognized topo I epitopes [20]. However, besides fragment F4 (AA 451-593; SEQ ID NO: 3) detected in all patient sera tested, fragment F1 (AA 5-30; SEQ ID NO: 2) was specifically recognized by a subset of dcSSc patients' sera, and fragment F8 (350-400; SEQ ID NO: 4) was recognized by SLE patients, indicating that these fragments could represent characteristic epitopes for dcSSc and SLE, respectively.

Thus, data obtained with a phage display based approach clearly demonstrate that the pattern of recognized epitopes is different between dcSSc, lcSSc and SLE patients. The finding of a common fragment recognized by all Topo I reactive patients' was located in the region of AA 451-593 (fragment F4), which is in agreement with previously published results [12,13]. However, according to the present results immunological assays, e.g. ELISA performed with fragment F4 may represent a more sensitive tool to detect anti-topo I autoantibodies than conventional ELISA systems using full length antigen.

In addition to an immunodominant part of topo I (fragment F4), two new regions have been identified which were previously not shown to be targeted by anti-topo I antibodies. dcSSc patients recognized several short fragments (spanning AA 5-145) at the N-terminal part of the molecule. Specifically, fragment F1 (AA 5-30) was recognized by a subset of dcSSc patients' sera, while fragment F8 was recognized by SLE patients.

Moreover, analysis of clinical data suggests that autoantibodies against fragment F1 may indicate the evolution of the disease in late stage dcSSc. This fragment F1 contains an experimentally proven granzyme B cleavage site [22]. Thus it is possible that in vivo cleavage of topo I by granzyme B released during T cell mediated cytotoxic responses results in the formation of a neo-antigenic determinant represented by fragment F1. In vitro assays using the full length antigen or the full length N-terminal domain may fail to detect antibodies recognizing these short epitopes.

Analysis of clinical data failed to demonstrate clear associations between anti-topo I antibody epitope specificity and clinical presentation of the disease. However, without being bound by theory, the difference in the duration of disease between anti-F1 antibody positive and negative dcSSc patients, together with findings of our longitudinal analysis, may indicate that the anti-topo I immune response is initiated against the immunodominant part of the molecule (fragment F4), and may target the N-terminal part later during the course of the disease. Thus autoantibodies against fragment F1 may represent a new marker of late stage dcSSc. The mechanism of this “epitope spreading” and factors which facilitate this in dcSSc remain to be further investigated.

Moreover, comparison of clinical data of the 4 F8 positive and the 261 F8 negative SLE patients suggested that SLE patients with antibody against fragment F8 have Raynaud's phenomenon and a milder presentation of the disease (lack of arthritis, central nervous system and kidney involvement).

In a preferred embodiment in the diagnostic method or use or in the kits of the invention the epitopes recognized by the autoantibodies are present on separate peptides. In a further preferred embodiment antibody standards may be prepared by usual immunological methods.

It is well within the skills of a person skilled in the art to detect autoantibody binding. It is immediately apparent for a skilled person that both kinetic methods as well as method characterizing binding affinity or avidity are applicable. For example, any of the methods below, without any limitation, are applicable:

an immunoassay, e.g. ELISA, RIA, lateral flow, immunoprecipitation,
a binding assay, e.g. Biacore, fluorescence quenching,
a spectrophotometric method, e.g. FT-IR, circular dichroism, NMR,
a physico-chemical method, e.g. calorimetry, ultracentrifugation etc.

In a preferred embodiment of the diagnostic method is carried out in the form of an immunoassay, like RIA or DELPHIA or preferably in an immunosorbent assay, like ELISA.

A detailed teaching can be found e.g. in the book Autoantibodies, by Yehuda Shoenfeld, Pier-Luigi Meroni, M. Eric Gershwin, Elsevier, second edition.

In certain embodiments the object may be to provide an assay which selectively differentiates among patient autoantibodies which a related to the disorder and other antibodies. Setting the sensitivity of the assay is well within the skills of a person skilled in the art, who will be able to find appropriate control peptides. Examples for such peptides are provided herein. A positive inner control is in fact F4 fragment itself. As a negative control other, non-immunogenic fragments or other proteins, like MBP can be used.

In a preferred variant IgM or IgG are measured as autoantibodies, provided that the patients are not deficient in any of these types.

The invention also relates to kits for performing the diagnostic methods as outlined above. These kits may comprise the parts as mentioned above or as useful in carrying out the methods outlined above. A kit necessarily comprises peptides carrying the epitope and at least instructions for use.

The invention is described below more specifically by non-limiting examples. The skilled person will understand, however, that other embodiments based on the idea and within the scope of the invention can be reduced in the practice based on knowledge in the art and his/her general knowledge. Moreover, the teaching of the cited references is specifically incorporated herein.

### EXAMPLES

### Methods

### Patients and Controls

From the 293 patients in our total scleroderma cohort, 59 SSc cases were selected which showed anti-topoisomerase antibody positivity on a conventional ELISA test (Hycor, Vienna, Austria) (34 of these patients were classified as having dcSSc and 25 were diagnosed lcSSc). Out of 265 SLE patients tested for the presence of anti-topo I antibodies with a conventional ELISA kit 8 showed positive reaction and were selected for the present study. 177 serum samples from 59 SSc patients (3 from each patient) and 24 serum samples from 8 SLE patients were obtained between 2004 and 2007 at 6 to 12 months intervals. The patients' clinical data were encoded using our standard protocol [8].

For controls 146 anti-topo I antibody negative serum samples from healthy women and men of various ages were used. Furthermore 110 age matched patients with different inflammatory rheumatic diseases (8 vasculitis, 40 seronegative spondylarthritis, 11 myositis, 11 Sjögren syndrome, 10 psoriatic arthritis, 20 rheumatoid arthritis, 10 polymyalgia rheumatica) were also investigated.

The study has been approved by the Ethical Committee of the Medical Center of the University of Pecs. Informed consent has been obtained from all patients and healthy individuals.

### Construction and Affinity Selection of Topo I Antigen Fragment Library

The coding region of full length human topo I (SEQ ID NO: 1) was amplified by PCR from cDNA reverse transcribed from total RNA. The PCR product was cloned into a T/A vector using the InsT/Aclone PCR Product Cloning Kit (Fermentas, Vilnius, Lithuania). Library construction was done using the lambdaD-bio phage display vector [9] with minor modifications as described previously [10]. The primary topo I library contained 2×10⁷ insert bearing independent clones; titer of the amplified library was 3×10¹¹/ml. Affinity selection of topo I antigen fragment library with 5 dcSSc, 6 lcSSc and 4 SLE patient derived IgG purified on protein G sepharose (Amersham Pharmacia, Uppsala, Sweden) was performed essentially as described [11]. After the third round of selection individual clones were picked up for further propagation and DNA sequencing.

### Expression of Recombinant Topo I Fusion Proteins

Selected fragments of topo I were expressed as recombinant maltose binding protein (MBP) fusion proteins using the pMAL Protein Fusion and Purification system (New England Biolabs, Ipswich, UK). cDNAs coding for AA 5-30 (F1), 69-92 (F2), 87-145 (F3), 450-600 (F4), 640-705 (F5), 170-290 (F6), 295-350 (F7), 350-400 (F8), 295-400 (F9) were amplified with PCR primers containing EcoRI and BamHI restriction sites and cloned into the pMal-c2 vector. Fusion proteins were purified from bacterial lysates with affinity chromatography on amylose resin according to the manufacturer's instruction (New England Biolabs, Ipswich, UK), and integrity of purified proteins was verified by SDS-PAGE on a 10% gel followed by Coomassie brilliant blue staining

### Elisa

96-well polystyrene plates (Nunc, Roskilde, Denmark) were coated with recombinant topo I fragments or with MBP in PBS at a concentration of 10 μg/ml. Plates were washed with wash buffer (PBS, 0.05% Tween-20) and blocked with 3% non-fat dry milk in wash buffer for 1 h. Serum samples were incubated in triplicates at 1:250 dilutions in wash buffer containing 2% non-fat dry milk for 1 h. Finally, the plate was incubated with HRP conjugated anti-human-IgG secondary antibody (Dako, Glostrup, Denmark) for 60 min. The reaction was developed with o-phenylenediamine (Sigma-Aldrich, Budapest, Hungary), and optical density (OD) was measured at 492 nm. In order to determine cut off values for further measurements sera of 146 healthy controls (previously tested negative for anti-topo I antibody with a commercial ELISA kit) were tested on all topo I fragments and MBP. Reactivity of healthy controls' sera with topo I fragments and MBP was shown to be minimal (OD₄₉₂ 0.019-0.032), and a cut off value of 0.1 have been chosen for further measurements.

### Immunoblots

Purified MBP fusion proteins or MBP (40 μg/ml) diluted 1:1 with SDS sample buffer were boiled for 10 minutes, separated on a 10% SDS-polyacrylamide gel and transferred to nitrocellulose membranes. After blocking with 5% non-fat dry milk (Bio-Rad, Budapest, Hungary) in wash buffer (100 mM NaCl, mM Tris-base pH 7.4, 0.1% Tween 20) for 1 h, membranes were incubated for 1 h with sera diluted 1:500 in 2% non-fat dry milk in wash buffer. After washing, HRP-conjugated anti-human-IgG diluted at 1:2000 was added for 1 hour. For detection of MBP fusion proteins membrane strips were first incubated with rabbit anti-MBP antibody (New England Biolabs, Ipswich, UK) (1:5000), followed by incubation with HRP-conjugated goat anti-rabbit antibody (1:2000). Membranes were developed with SuperSignal West Pico Chemiluminescent (Pierce, Rockford, USA) substrate and exposed to x-ray films.

### Statistical Analysis

Categorical data were analyzed by the Chi-square test. To investigate the possible differences between patient groups, frequency and mean values of continuous variables were tested by Student's t test. Spearman's rank correlation coefficient was used to examine the relationship between the values of optical density and continuous variables. A p value less than 0.05 was considered statistically significant. Statistical analyses were conducted using SPSS statistical software package.

Thus, while the present diagnostic method may not be applicable in each case, if the patient has topo-I reactive antibodies, it can safely differentiate between at least dcSSc and SLE.

### Results

Epitope Mapping of Anti-Topo I Antibodies with Phage Displayed Topo I Library

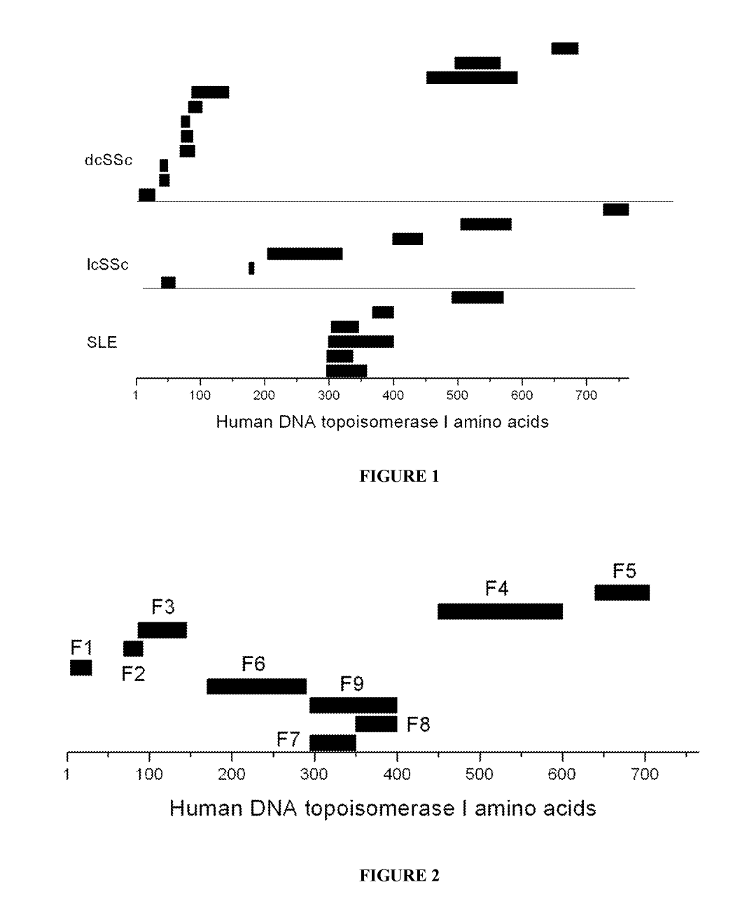

For identification of epitopes recognized by anti-topo I antibodies we have constructed a topo I antigen fragment library displayed on bacteriophage lambda, and subsequently screened this library with individual IgGs purified from sera of anti-topo I positive patients (5 dcSSc, 6 lcSSc and 4 SLE patient). After the third round of affinity selection inserts of 60 clones (30 from each patient group) were sequenced. Alignment of deduced amino acid sequences with human topo I showed that the pattern of recognized epitopes is different between dcSSc, lcSSc and SLE patients (FIG. 1).

### Recognition of Recombinant Topo I Fusion Proteins by Anti-Topo I Antibodies

In order to verify results obtained by affinity selection of the topo I antigen fragment library we have constructed recombinant topo I-MBP fusion proteins. On the basis of fragments identified by library selection nine fusion proteins have been constructed and expressed (FIG. 2). Recognition of these fusion proteins was tested with sera of 67 anti-topo I antibody positive patients (34 dcSSc, 25 lcSSc and 8 SLE) including those which have been used for library selection by ELISA. The results are summarized in table 1.

TABLE 1
Recognition frequencies of recombinant topoisomerase I
(topo I) fragments determined by
ELISA using anti-topo I antibody
positive patients′ sera

| Topo I fragments | dcSSc | | lcSSc | | SLE | |
|---|---|---|---|---|---|---|
| (amino acids) | (n = 34) | | (n = 25) | | (n = 8) | |
| F1 (5-30) | 9 | (26%) | 1 | (4%) | 0 | |
| F2 (69-92) | 1 | (3%) | 1 | (4%) | 0 | |
| F3 (87-145) | 1 | (3%) | 0 | | 0 | |
| F4 (450-600) | 34 | (100%) | 25 | (100%) | 8 | (100%) |

| F5 (640-705) | 2 | (6%) | 0 | | 0 |
|---|---|---|---|---|---|
| F6 (170-290) | 2 | (6%) | 2 | (8%) | 0 |

| | | | | |
|---|---|---|---|---|
| F7 (295-400) | 0 | 0 | 1 | (12%) |
| F8 (350-400) | 0 | 0 | 4 | (50%) |
| F9 (295-350) | 0 | 0 | 1 | (12%) |

Numbers indicate individual patients positive for the given fragments, numbers in parentheses indicate percentage of positive sera (dcSSc: diffuse cutaneous systemic sclerosis; lcSSc: limited cutaneous systemic sclerosis; SLE: systemic lupus erythematosus).

Fragment F4 (AA 450-600) was recognized by all of the 67 patients' sera. Fragment F1 (AA 5-30) was recognized by 9 of 34, 1 of 25 and 0 of 8 dcSSc, lcSSc and SLE patients, respectively. Fragment F8 (AA 350-400) was recognized by 4 of 8 SLE patients and none of the SSc patients.

### Longitudinal Analysis of Topo I Epitope Reactivity

To determine whether antibody responses against fragments F1, F4 and F8 remain constant over time, we measured antibody reactivity against these fragments by ELISA in 3 serial serum samples of each patient. Results of ELISA were confirmed by western blot in 10 F1 positive patients, 10 randomly chosen F4 positive patients and 4 F8 positive patients. Each serum sample was also examined by conventional anti-Scl-70 ELISA.

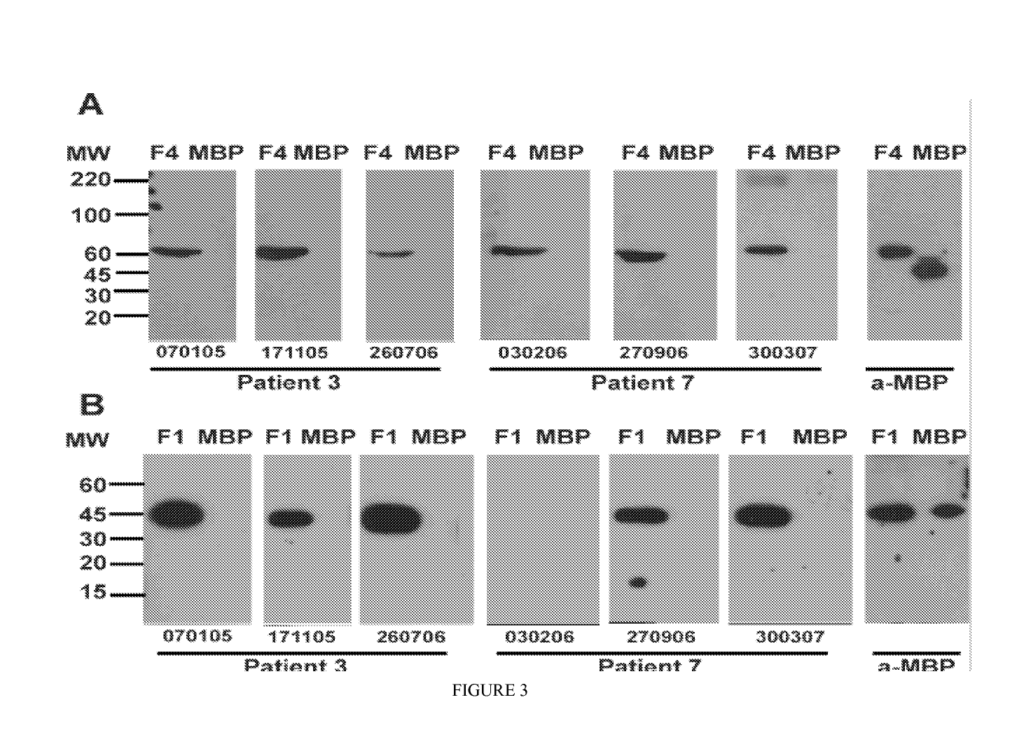

Longitudinal analysis showed that reactivity to fragment F4 was stable in 61 cases (94%). 18 out of the 67 patients had at least one serum sample with no antibody response against topo I measured by the conventional anti-Scl-70 ELISA. Results of ELISA with fragment F4 were confirmed by western blot, which showed that each sera positive for F4 reactivity in ELISA were also positive in western blot (FIG. 3A).

The reactivity to F1 fragment varied over time. In 4 cases the earliest serum samples did not have any detectable antibodies against F1, but the immunoreaction became positive and stronger over time. In one case reactivity against F1 appeared in the second sample but was absent in the following one. All serum samples of the remaining 4 dcSSc patients were positive for anti-F1 antibody. Results of ELISA were confirmed by western blot, which showed a perfect correlation of results obtained by the two methods (FIG. 3B).

Among the 4 SLE patients positive for antibody against F8 the reactivity was stable in one patient, and changed in the remaining three patients.

### Clinical Findings

Statistical analyses of clinical data (extent of skin involvement, hand contractures, azotemia and/or malignant hypertension, cardiac involvement, dysmotility and stricture/dilatation of esophagus, extent of lung fibrosis, forced vital capacity) showed no association between anti-topo I antibody epitope specificity and clinical presentation of SSc. However, there was a significant difference between the F1 negative and F1 positive groups of SSc patients in average age (F1 negative (number of patients: 49): 54.8±13.5 years; F1 positive (number of patients: 10): 63.9±9.4 years; p=0.048) and the duration of the disease (F1 negative (number of patients: 49): 10.0±7.3 years; F1 positive (number of patients: 10): 17.1±12.9 years; p=0.019). Moreover, average age of dcSSc patients possessing sera positive for F1 fragment was significantly higher compared to the patients showing no detectable levels of antibody against F1 (F1 negative (number of patients: 25): 51.9±14.3 years; F1 positive (number of patients: 9): 63.7±10.0 years; p=0.03).

To investigate whether antibodies against peptide F1 were specific for SSc patients and the appearance of them is not merely a consequence of aging, sera from 64 age matched (average age: 62.4±5.4 years) healthy controls were tested for antibodies against F1. Only one serum sample was found positive. To test whether the presence of antibodies against fragment F1 is specific for SSc, sera from 110 age matched (average age: 65.5±4.8 years) patients with different connective tissue diseases were also tested and only 4 serum samples (2 seronegative spondylarthritis, 1 myositis, 1 Sjögren syndrome) were found positive.

Comparison of clinical data of the 4 F8 positive and the 261 F8 negative SLE patients suggested that SLE patients with antibody against fragment F8 have Raynaud's phenomenon and a milder presentation of the disease (lack of arthritis, central nervous system and kidney involvement).

In summary, it has been demonstrated that in addition to an immunodominant part of topo I, sera of patients with dcSSc, lcSSc and SLE recognize distinct topo I epitopes. We have shown that recognition of the majority of fragments is characteristic for the individual patient, instead of being characteristic for the given disease subgroup. However, fragment F1 (AA 5-30) was specifically recognized by a subset of dcSSc patients' sera, and fragment F8 (350-400) was recognized by SLE patients, indicating that fragment F1 and F8 could represent characteristic epitopes for dcSSc and SLE, respectively.

Funding: National Health Fund (ETT: 32/KO/2004)

### REFERENCES

1. Varga J, Abraham D. Systemic sclerosis: a prototypic multisystem fibrotic disorder. J Clin Invest 2007; 117:557-67.
2. Okano Y. Antinuclear antibody in systemic sclerosis (scleroderma). Rheum Dis Clin North Am 1996; 22:709-35.
3. Steen V D, Powell D L, Medsger T A Jr. Clinical correlations and prognosis based on serum autoantibodies in patients with systemic sclerosis. Arthritis Rheum 1988; 31:196-203.
4. Kuwana M, Kaburaki J, Okano Y, Tojo T, Homma M. Clinical and prognostic associations based on serum antinuclear antibodies in Japanese patients with systemic sclerosis. Arthritis Rheum 1994; 37:75-83.
5. LeRoy E C, Black C, Fleischmajer R, Jablonska S, Krieg T, Medsger T A Jr, et al. Scleroderma (systemic sclerosis): classification, subsets and pathogenesis. J Rheumatol 1988; 15:9202-5.
6. Gussin H A, Ignat G P, Varga J, Teodorescu M. Anti-topoisomerase I (anti-Scl-70) antibodies in patients with systemic lupus erythematosus. Arthritis Rheum 2001; 44:376-83.
7. Hamidou M A, Audrain M A, Masseau A, Agard C, Moreau A. Anti-topoisomerase I antibodies in systemic lupus erythematosus as a marker of severe nephritis. Clin Rheumatol 2006; 25:542-3.
8. Czirják L, Kumánovics G, Varjú C, Nagy Z, Pákozdi A, Szekanecz Z et al. Survival and causes of death in 366 Hungarian patients with systemic sclerosis. Ann Rheum Dis. 2008; 67:59-63.
9. Ansuini H, Cicchini C, Nicosia A, Tripodi M, Cortese R, Luzzago A. Biotin-tagged cDNA expression libraries displayed on lambda phage: a new tool for the selection of natural protein ligands. Nucleic Acids Res 2002; 30: e78.
10. Czömpöly T, Olasz K, Simon D, Nyárády Z, Pálinkás L, Czirják L et al. A possible new bridge between innate and adaptive immunity: Are the anti-mitochondrial citrate synthase autoantibodies components of the natural antibody network? Mol Immunol 2006; 43:1761-8.
11. Santini C, Brennan D, Mennuni C, Hoess R H, Nicosia A, Cortese R et al. Efficient display of an HCV cDNA expression library as C-terminal fusion to the capsid protein D of bacteriophage lambda. J Mol Biol 1998; 282:125-35.
12. D'Arpa, P, White-Cooper H, Cleveland D W, Rothfield N F, Earnshaw W C. Use of molecular cloning methods to map the distribution of epitopes on topoisomerase I (Scl-70) recognized by sera of scleroderma patients. Arthritis Rheum 1990; 33:1501-11.
13. Verheijen, R, Van den Hoogen F, Beijer R, Richter A, Penner E, Habets W J et al. A recombinant topoisomerase I used for autoantibody detection in sera from patients with systemic sclerosis. Clin Exp Immunol 1990; 80:38-43.
14. Piccinini G, Cardellini E, Reimer G, Arnett F C, Durban E. An antigenic region of topoisomerase I in DNA polymerase chain reaction-generated fragments recognized by autoantibodies of scleroderma patients. Mol Immunol 1991; 28:333-9.
15. Cram D S, Fisicaro N, McNeilage L J, Coppel R L, Harrison L C. Antibody specificities of That and Australian scleroderma sera with topoisomerase I recombinant fusion proteins. J Immunol 1993; 151:6872-81.
16. Kuwana M, Kaburaki J, Mimori T, Tojo T, Homma M. 1993. Autoantigenic epitopes on DNA topoisomerase I: clinical and immunogenetic associations in systemic sclerosis. Arthritis Rheum 1993; 36:1406-13.
17. Seelig H P, Schroter H, Ehrfeld H, Renz M. Autoantibodies against topoisomerase I detected with the natural enzyme and overlapping recombinant peptides. J Immunol Methods 1993; 165:241-52.
18. Kato T, Yamamoto K, Takeuchi H, Okubo M, Hara E, Nakada S, et al. Identification of a universal B cell epitope on DNA topoisomerase I, an autoantigen associated with scleroderma. Arthritis Rheum 1993; 36:1580-7.
19. Kuwana M, Kaburaki J, Medsger T A Jr, Wright T M. An immunodominant epitope on DNA topoisomerase I is conformational in nature: heterogeneity in its recognition by systemic sclerosis sera. Arthritis Rheum 1999; 42:1179-88.
20. Henry P A, Atamas S P, Yurovsky V V, Luzina I, Wigley F M, White B. Diversity and plasticity of the anti-DNA topoisomerase I autoantibody response in scleroderma. Arthritis Rheum 2000; 43:2733-42.
21. Hu P Q, Fertig N, Medsger T A Jr, Wright T M. Molecular recognition patterns of serum anti-DNA topoisomerase I antibody in systemic sclerosis. J Immunol 2004; 173:2834-41.
22. Casciola-Rosen L, Andrade F, Ulanet D, Wong W B, Rosen A. Cleavage by granzyme B is strongly predictive of autoantigen status: implications for initiation of autoimmunity J Exp Med 1999; 190:815-26.
23. Redinbo, M. R., Stewart, L., Kuhn, P., Champoux, J. J. and Hol, W. G. J. 1998. Crystal structures of human topoisomerase I in covalent and noncovalent complexes with DNA. Science 279:1504.

1.-5. (canceled)

6. A diagnostic kit for use in the diagnosis of a systemic autoimmune disorder, said kit comprising one or more peptides as defined in claim 9, said one or more peptide(s) separately or simultaneously comprising at least two of the following epitopes:
a) an epitope having the immunological property of an at least 20 amino acids long segment of the portion of a DNA Topoisomerase I spanning amino acids 5 to 30,
b) an epitope having the immunological property of an at least 20 amino acids long segment of the portion of a DNA Topoisomerase I spanning amino acids 350 to 400, and
c) an epitope having the immunological property of an at least 20 amino acids long segment of the portion of a DNA Topoisomerase I spanning amino acids 451 to 593.

7. The diagnostic kit according to claim 6, said kit comprising
at least one peptide selected from
a) a peptide comprising an epitope having the immunological property of an at least 20 amino acids long segment of the portion of a DNA Topoisomerase I spanning amino acids 5 to 30,
b) a peptide comprising an epitope having the immunological property of an at least 20 amino acids long segment of the portion of a DNA Topoisomerase I spanning amino acids 350 to 400, and
a reference peptide comprising an epitope having the immunological property of an at least 20 amino acids long segment of the portion of a DNA Topoisomerase I spanning amino acids 451 to 593; and optionally
means for detecting binding of patient autoantibodies to a peptide epitope.

8. The diagnostic kit according to claim 7, wherein
the peptide according to a) is a peptide fragment of said DNA Topoisomerase I spanning amino acids 5 to 30,
the peptide according to b) is a peptide fragment of said DNA Topoisomerase I spanning amino acids 350 to 400, and
the peptide according to c) is a peptide fragment of said DNA Topoisomerase I spanning amino acids 451 to 593.

9. A diagnostic method useful for differential diagnosis of systemic autoimmune disorders comprising the steps of
i) contacting a sample obtained from a patient with one or more peptide(s) comprising at least one of the following epitopes:
a) an epitope having the immunological property of an at least 20 amino acids long segment of the portion of said DNA Topoisomerase I spanning amino acids 5 to 30,
b) an epitope having the immunological property of an at least 20 amino acids long segment of the portion of said DNA Topoisomerase I spanning amino acids 350 to 400
said one or more peptide(s) having an amino acid sequence which is at least 70% identical with the amino acid sequence of a peptide fragment of a wild type DNA Topoisomerase I,
wherein
if binding of autoantibodies of said sample to epitope according to a) is detected, it is considered as indicative of diffuse cutane systemic scleroderma (dsSSC) in said patient,
if binding of autoantibodies of said sample to epitope according to b) is detected, it is considered as indicative of systemic lupus erithematosus (SLE) in said patient.

10. The diagnostic method according to claim 9, wherein the one or more isolated peptide(s) also comprise
c) an epitope having the immunological property of the at least 20 amino acids long segment of the portion of said DNA Topoisomerase I spanning amino acids 451 to 593, and wherein
if binding of autoantibodies to epitope according to a) and c) is detected it is considered as indicative of diffuse cutane systemic scleroderma (dsSSC) in said patient,
if binding of autoantibodies to epitope according to b) and c) is detected, it is considered as indicative of systemic lupus erithematosus (SLE) in said patient.

11. The diagnostic method of claim 10, wherein a kit is used, said kit comprising one or more peptide(s),
said one or more peptide(s) separately or simultaneously comprising at least two of the following epitopes:
a) an epitope having the immunological property of an at least 20 amino acids long segment of the portion of a DNA Topoisomerase I spanning amino acids 5 to 30,
b) an epitope having the immunological property of an at least 20 amino acids long segment of the portion of a DNA Topoisomerase I spanning amino acids 350 to 400, and
c) an epitope having the immunological property of an at least 20 amino acids long segment of the portion of a DNA Topoisomerase I spanning amino acids 451 to 593.

12. The diagnostic method of claim 9, wherein the sample is a blood sample.

13. The method of claim 12, wherein the sample is a serum sample.

14. The method of claim 9 wherein the epitope of said peptide is different in not more than 10 amino acids from the respective epitope of the wild type sequence.

15. The method of claim 9 wherein the epitope of said peptide is different in not more than 5 amino acids from the respective epitope of the wild type sequence.

16. The method of claim 10, wherein
the epitope according to a) is an at least 20 amino acids long segment of the portion of said DNA Topoisomerase I spanning amino acids 5 to 30,
the epitope according to b) is an at least 20 amino acids long segment of the portion of said DNA Topoisomerase I spanning amino acids 350 to 400, and
the epitope according to c) is an at least 20 amino acids long segment of the portion of said DNA Topoisomerase I spanning amino acids 451 to 593.

17. The method of claim 9 wherein the epitopes are present on separate peptides.

18. The method of claim 9 wherein said one or more peptide(s) has an amino acid sequence which is at least 90% identical with the amino acid sequence of a peptide fragment of a wild type DNA Topoisomerase I.

19. The method of claim 10, wherein
if binding of autoantibodies to epitope according to a) and c) is detected, and binding of autoantibodies of said patient sample to epitope according to b) is not detected, it is considered as indicative of diffuse cutane systemic scleroderma (dsSSC) in said patient,
if binding of autoantibodies to epitope according to b) and c) is detected, and binding of autoantibodies of said patient sample to epitope according to a) is not detected, it is considered as indicative of systemic lupus erithematosus (SLE) in said patient.

20. The method of claim 9 wherein
if binding of autoantibodies of said sample to epitope according to a) is detected, it is considered as indicative of a late onset of diffuse cutane systemic scleroderma (dsSSC) in said patient,
if binding of autoantibodies of said sample to epitope according to b) is detected, it is considered as indicative of systemic lupus erithematosus (SLE) with Raynaud's phenomenon in said patient.

21. The method of claim 10, wherein
if binding of autoantibodies to epitope according to a) and c) is detected, and binding of autoantibodies of said patient sample to epitope according to b) is not detected, it is considered as indicative of the late onset of diffuse cutane systemic scleroderma (dsSSC) in said patient,
if binding of autoantibodies to epitope according to b) and c) is detected, and binding of autoantibodies of said patient sample to epitope according to a) is not detected, it is considered as indicative of systemic lupus erithematosus (SLE) with Raynaud's phenomenon in said patient.


SEQUENCE LISTING (4 sequences)

SEQ ID NO: 1 - protein, 765 aa, Homo sapiens
MSGDHLHNDSQIEADFRLNDSHKHKDKHKDREHRHKEHKKEKDREKSKHSNSEHKDSEKKHKEKEKTKHKDGSSEKHKDKHKDRDKEKRKEEKVRASGDAKIKKEKENGFSSPPQIKDEPEDDGYFVPPKEDIKPLKRPRDEDDADYKPKKIKTEDTKKEKKRKLEEEEDGKLKKPKNKDKDKKVPEPDNKKKKPKKEEEQKWKWWEEERYPEGIKWKFLEHKGPVFAPPYEPLPENVKFYYDGKVMKLSPKAEEVATFFAKMLDHEYTTKEIFRKNFFKDWRKEMTNEEKNIITNLSKCDFTQMSQYFKAQTEARKQMSKEEKLKIKEENEKLLKEYGFCIMDNHKERIANFKIEPPGLFRGRGNHPKMGMLKRRIMPEDIIINCSKDAKVPSPPPGHKWKEVRHDNKVTWLVSWTENIQGSIKYIMLNPSSRIKGEKDWQKYETARRLKKCVDKIRNQYREDWKSKEMKVRQRAVALYFIDKLALRAGNEKEEGETADTVGCCSLRVEHINLHPELDGQEYVVEFDFLGKDSIRYYNKVPVEKRVFKNLQLFMENKQPEDDLFDRLNTGILNKHLQDLMEGLTAKVFRTYNASITLQQQLKELTAPDENIPAKILSYNRANRAVAILCNHQRAPPKTFEKSMMNLQTKIDAKKEQLADARRDLKSAKADAKVMKDAKTKKVVESKKKAVQRLEEQLMKLEVQATDREENKQIALGTSKLNYLDPRITVAWCKKWGVPIEKIYNKTQREKFAWAIDMADEDYEF

SEQ ID NO: 2 - protein, 26 aa, Artificial Sequence
  Fragment aa 5-30 of human topoisomerase I
HLHNDSQIEADFRLNDSHKHKDKHKD

SEQ ID NO: 3 - protein, 142 aa, Artificial
  Fragment aa 451-593 of human topoisomerase I
KCVDKIRNQYREDWKSKEMKVRQRAVALYFIDKLALRAGNEKEEGETADTVGCCSLRVEHINLHPELDGQEYVVEFDFLGKDSIRYYNKVPVEKRVFKNLQLFMENKQPEDDLFDRLNTGILNKHLQDLMEGLTAKVFRTYN

SEQ ID NO: 4 - protein, 52 aa, Artificial
  Fragment aa 350-400 of human topoisomerase I
IANFKIEPPGLFRGRGNHPKMGMLKRRIMPEDIIINCSKDAKVPSPPPGHKW